(19) United States
(12) Patent Application Publication
(10) Pub. No.: US 2010/0004642 A1
(43) Pub. Date: Jan. 7, 2010

(54) Selectively bendable laser fiber for surgical laser probe

(76) Inventor: Christopher F. Lumpkin, Steamboat Springs, CO (US)

Correspondence Address: Kenneth Watov;WATOV & KIPNES, P.C., P.O. Box 247, Princeton Junction, NJ, 08550, US

(21) Appl. No.: 12/459,215

(22) Filed: Jun. 29, 2009

Related U.S. Application Data
(60) Provisional application No. 61/133,794, filed on Jul. 2, 2008.

(51) Int. Cl. A61F 9/008; A61B 18/22

(52) U.S. Cl. 606/4; 606/16

(57) ABSTRACT

A surgical laser probe includes a handpiece having a proximal end for receiving an optical fiber into a centrally located throughhole, and a distal end through which the centrally located throughhole extends and varies in diameter. An end portion of a stainless steel tube is rigidly secured within the hole in the distal end of the handpiece. An end portion of a polyimide bendable tube is secured to the free end of the stainless steel tube. A slideable finger pad is installed in a slotway of the handpiece and rigidly secured to one end of a Nitinol wire. The optical fiber is extended through the handpiece, the stainless steel tube, and into the bendable tube, with the free end of the optical fiber terminated at the free end of the polyimide bendable tube. As the finger pad is slid toward the distal end of the handpiece, the free end of the Nitinol wire is increasingly moved into the bendable tube for increasingly bending the bendable tube and optical fiber away from the longitudinal axis of the stainless steel tube.

### RELATED APPLICATION

This Application is related to and take priority from Provisional Serial No. 61/133,794, filed Jul. 2, 2008, the teachings of which are incorporated herein to the extent they do not conflict herewith.

### FIELD OF THE INVENTION

The present invention is generally related to laser probes, and is more particularly related to laser probes that provide a vitreoretinal surgeon the ability to position the distal end of a laser fiber internally in a patient's eye, and to selectively bend the fiber's distal end to treat a targeted area within the eye.

### BACKGROUND OF THE INVENTION

There is a need to provide improved laser probes for permitting vitreoretinal surgeons to invasively treat targeted internal portions of a patient's eye in the most time efficient manner possible.

### SUMMARY OF THE INVENTION

An object of the invention is to provide a improved laser probe for use by vitreoretinal surgeons.

The improved laser probe includes a handpiece having one end from which a fixed length of optical fiber extends partly within a fixed length of rigid tubing, the latter being secured at its proximal end to the one end of the handpiece. A fixed length free portion of the optical fiber within a bendable plastic tubing extends from a distal end of the rigid tubing. Adjustable means are provided in the handle for permitting a surgeon to selectively move bending means from the rigid tubing into different positions within the bendable plastic tubing, for bending the free end of the optical fiber a desired amount.

### BRIEF DESCRIPTION OF THE DRAWINGS

Various embodiments of the present invention are described in detail with reference to the accompanying drawings in which like items are designated by the same reference designation, wherein:

FIG. 1A is a side-elevational view of the laser probe, for one embodiment of the invention;

FIG. 1B is an enlarged cross-sectional view of the area taken along 1B-1B of FIG. 1A;

FIG. 2A is a side-elevational view of the laser probe for another embodiment of the invention;

FIG. 2B is an enlarged cross-sectional view of the area taken along 2B-2B of FIG. 2A;

FIG. 3 is a detailed exploded cutaway view of a front portion of a handpiece and associated components for an embodiment of the present invention; and

FIG. 4 is a detailed exploded cutaway view of a middle portion of a handpiece and associated components for an embodiment of the invention.

### DETAILED DESCRIPTION OF THE INVENTION

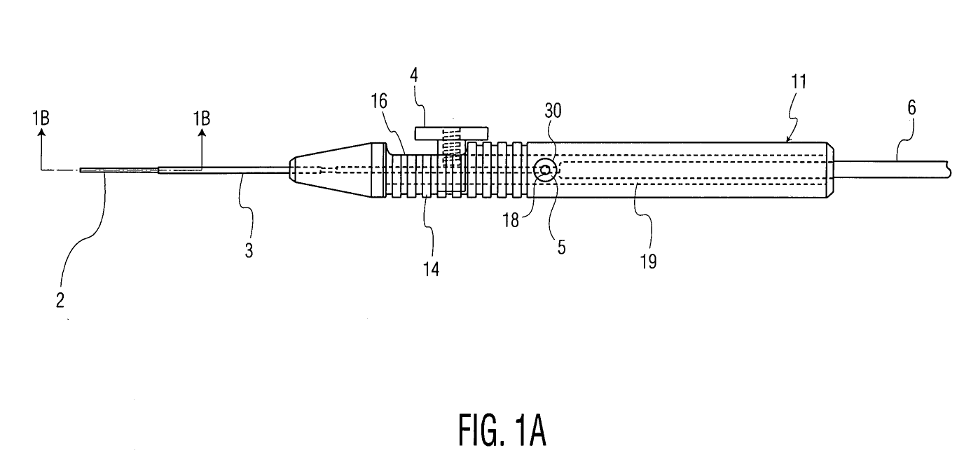

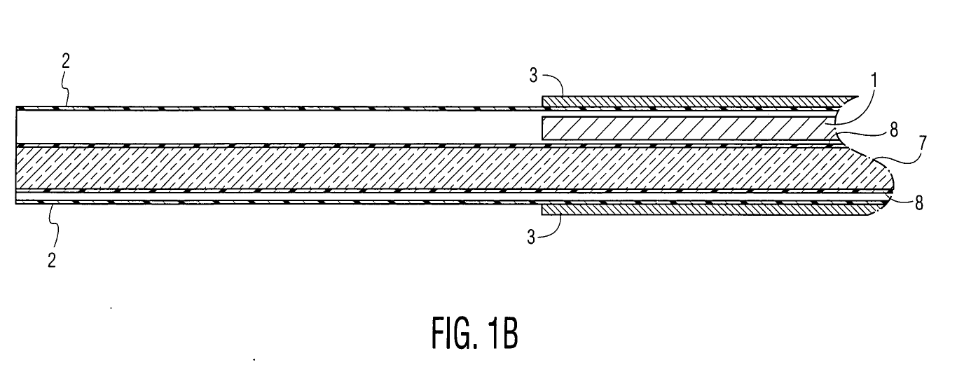

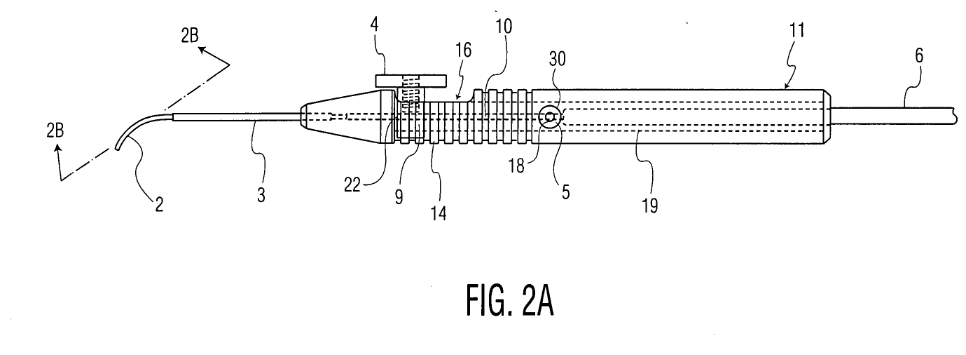

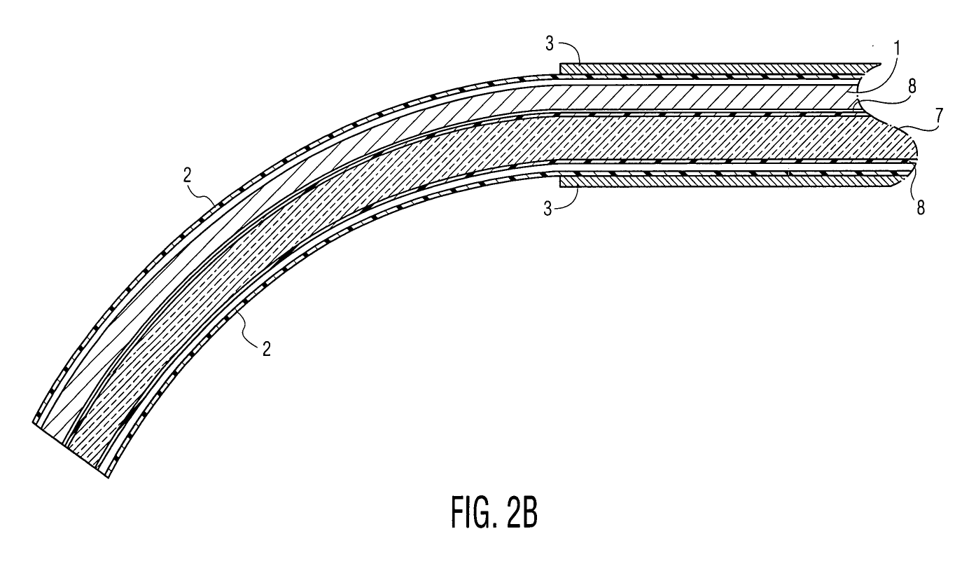

With reference to FIGS. 1A, 1B, 2A and 2B, the present laser probe includes an outer stainless steel tube or needle 3 that has its proximal end rigidly connected within the central bore of a handle or handpiece 11. A polyimide plastic tube 2 is contained within the stainless steel tube 3, and has a predetermined distal portion extending from the distal end of the stainless steel tube or needle 3. An optical fiber 7 extends through the handle 11, through the polyimide plastic tubing 2, and is terminated at the distal end of the plastic tubing 2. A preformed Nitinol wire 1 can be selectively positioned or located within the plastic tubing 2 adjacent the optical fiber 7. The proximal end of the Nitinol wire 1 is rigidly connected to a slidable finger pad 4 within the handle, in a manner permitting the distal end of the wire 1 to be moved to a retracted position proximate the distal end of the outer sleeve or the stainless steel needle from which the distal end of the plastic tubing 2 protrudes. The finger pad 4 can be selectively moved to move the distal end of the Nitinol Wire 1 into the end of the plastic tubing 2 extending from the needle 3, thereby causing the extended end of the plastic tubing 2 to gradually bend away from the longitudinal axis of the handle 11 and associated tubing 3, thereby causing the optical fiber 7 to bend with the plastic tubing 2. As the Nitinol wire 1 is extended further into the extended end of the plastic tubing 2, the tubing 2 and optical fiber 7 can gradually be bent 900 or more from the longitudinal axis of the stainless steel tube or needle 3. Note that the laser or optical fiber 7 is fixed in the length of its extension from the straight and rigid stainless steel tube 3. Also, circular ridges 14 are provided proximate the finger pad 4 and its associated slide slot 16. A hole 30 is located in handle 11, as shown, for receiving a wirelock tube 5. As will be described in greater detail below, a set screw 18 is installed in tube 5 for rigid securement to another tube 10.

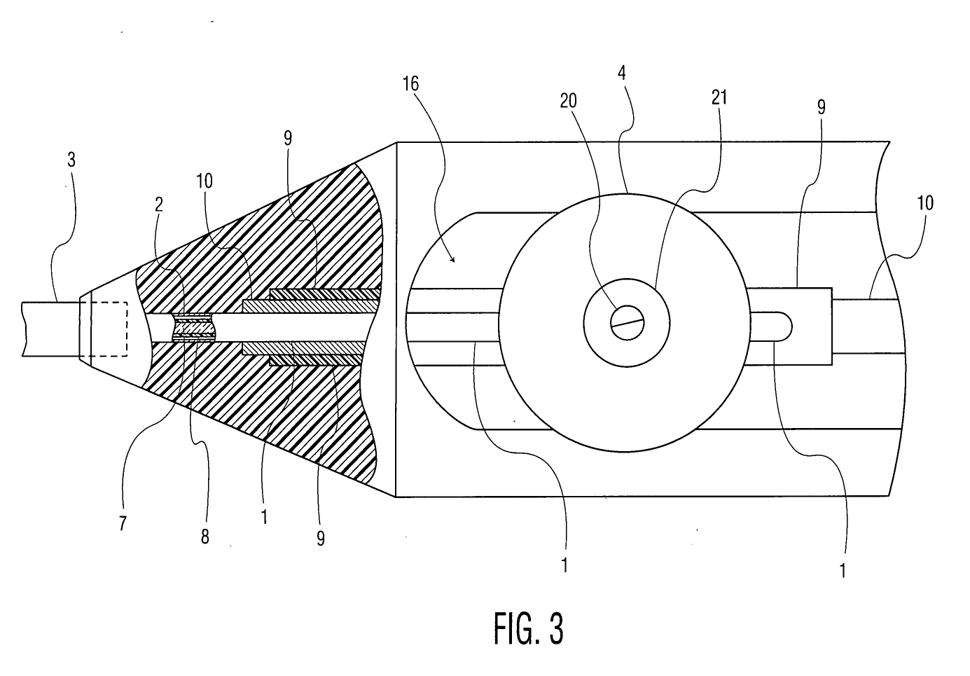

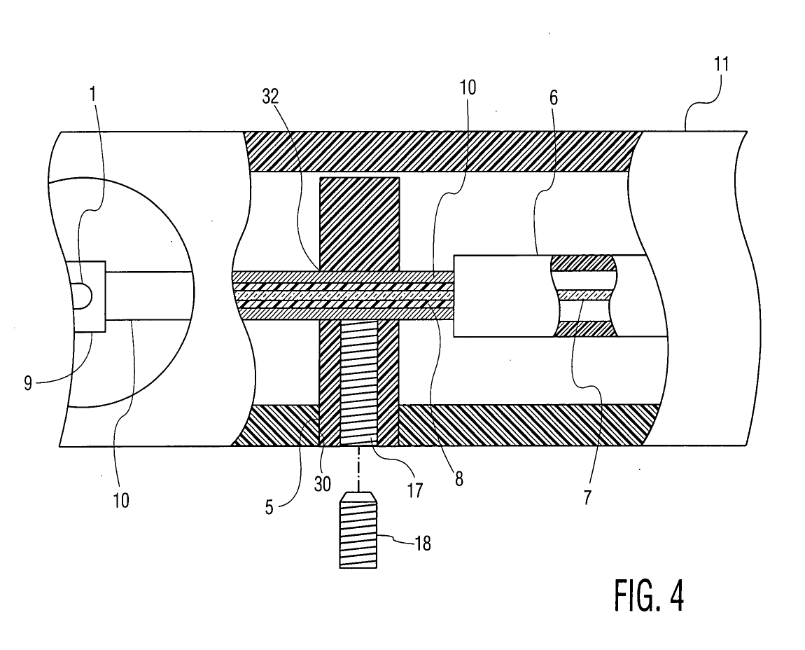

In one embodiment, as shown in FIG. 3, a length of tubing 9 encloses a portion of the laser fiber or optical fiber 7 within polyimide tube 8, and are secured together via use of an appropriate glue, adhesive, or epoxy. As shown in FIG. 4, the wire lock tube 5 secures via set screw 18 the tubing 10 with the optical fiber 7 and polyimide tube 8 in place, within a transverse hole 32 in tube 5. The polyimide bendable tubing 2 has an end portion secured within a distal end portion of the stainless steel (SS) tube 3 via use of an appropriate glue or epoxy. Alternatively, the tubing 2 end portion can be glued or epoxyed to an outside distal portion of the SS tube 3. The polyimide tubing 8 protects the optical fiber 7 from direct contact with the Nitinol wire 1, as the latter is slid back and forth via movement of finger pad or slide button 4. The sheath 6 covering the optical fiber 7 before entry into handpiece 11 and for a portion of the optical fiber 7 within the circular cavity 19 of handpiece 11, provides protection for the optical fiber 7 from external damage.

As shown in FIG. 3, a set screw 20 is installed in a threaded hole 21 of slide button or finger pad 4. The Nitinol wire 1 is positioned on the outside wall of tube 9, within a transverse through hole 22 (see FIG. 2A) in slide button 4. The Nitinol wire 1 is further positioned between the securement end of set screw 20 and tube 9. Set screw 20 is tightened to secure the Nitinol wire 1 and tube 9 to slide button 4.

The tube 9 is slideably mounted over a portion of a tube 10 within the slot 16 and a portion of cavity 19 of handpiece 11. Optical fiber 7 is passed through and has a portion retained within tube 10 (see FIGS. 2A, 3, and 4). One end of tube 10 is rigidly secured with handpiece 11 by a set screw 18 screwed into a threaded hole 17 in the side of handpiece 11, thereby providing wirelock 5. Note also that the inside walls of tube 9 are coated with a low-friction material such as polyimide, Teflon®, or any other suitable material, to enhance the sliding of tube 9 over tube 10.

Although various embodiments of the invention have been shown and described, they are not meant to be limiting. Those of skill in the art may recognize certain modifications to these embodiments, which modifications are meant to be covered by the spirit and scope of the appended claims.

What is claimed is:

1. A surgical laser probe comprising:
an optical fiber;
an elongated handpiece including a proximal end with a centrally located hole therethrough that extends therefrom through a major back portion to an open slotway, for receiving said optical fiber, the centrally located hole further extending through the slotway, and a distal end portion of the handpiece;
a straight length of rigid first tubing having a free end, and an opposing end portion rigidly secured within the distal end of the handpiece;
a predetermined length of bendable second tubing having a free end, and a portion of an opposing end secured within a portion of the free end of the first tubing;
said optical fiber extending through said handle to the free end of said second tubing, the optical fiber being fixed in position;
a finger pad slideably retained within said open slotway of said handpiece;
a Nitinol wire having one end rigidly secured to said finger pad, a portion of said Nitinol wire being positioned within said second tubing, whereby when said finger pad is positioned in said slotway to the furthest extent from the distal end of the handpiece, said Nitinol wire has a free end positioned in alignment with the free end of the first tubing, whereby as the finger pad is moved toward the distal end of the handpiece, the free end of said Nitinol Wire is moved increasingly more into and toward the free end of said second tubing, causing said second tubing and its contained optical fiber free end portion to increasingly bend away from the longitudinal axis of said first tubing and handpiece.

2. The laser probe of claim 1, wherein said first tubing consists of stainless steel.

3. The laser probe of claim 1, wherein said second tubing consists of polyimide.

4. The laser probe of claim 1, wherein said optical fiber is covered by a protective tubing at least between said finger pad and the free end of said optical fiber.

5. The laser probe of claim 4, wherein said protective tubing consists of polyimide.
